(19) United States
(12) Patent Application Publication
(10) Pub. No.: US 2012/0150061 A1
(43) Pub. Date: Jun. 14, 2012

(54) Sensor for Detecting Cancerous Tissue and Method of Manufacturing the Same

(75) Inventors: Kyung Hwa YOO, Seoul (KR); Sun Mi LEE, Seoul (KR); Ri Mi LEE, Daegu (KR)

(73) Assignee: INDUSTRY-ACADEMIC COOPERATION FOUNDATION, YONSEI UNIVERSITY, Seoul (KR)

(21) Appl. No.: 13/285,869

(22) Filed: Oct. 31, 2011

(30) Foreign Application Priority Data
Nov. 2, 2010 (KR) 10-2010-0108269

(51) Int. Cl. A61B 5/053; H05K 3/10

(52) U.S. Cl. 600/547; 29/846

(57) ABSTRACT

Disclosed herein are a sensor for detecting cancerous tissue, a method of manufacturing the same, and a method of monitoring the presence and status of cancerous tissue in real time. The sensor for detecting cancerous tissue includes a board, one or more pairs of needle electrodes, and an output unit. The needle electrodes are formed on the board, and obtain electrical signals from tissue. The output unit outputs the electrical signals, obtained from the electrodes, to the outside.

### BACKGROUND OF THE INVENTION

1. Field of the Invention

The present invention relates generally to a sensor for detecting cancerous tissue and a method of manufacturing the same.

2. Description of the Related Art

Cancer is the most frequent cause of death for Koreans. According to a statistics released in 2008, gastric cancer is the third-leading cause of death, and the number of colorectal cancer cases has increased by 101.4% compared to 1998. One simple and commonly used method of making an early diagnosis of cancer, such as gastric and colorectal cancer, is endoscopy.

The wide use of endoscopy is allowing for gastric and colorectal cancers to be detected early, and thus the number of deaths due to cancer has drastically decreased. This indicates that the danger of cancer may be greatly alleviated only by observing the inside of the stomach or the intestines with the eyes. However, one of the problems with endoscopy is that it is not possible to detect the presence of cancer if the size of the cancerous tissue is too small to be seen with the eyes. Currently, if cancer is suspected when a patient is undergoing endoscopy, tissue may be collected from an organ of the patient, cultured and then subjected to a separate tissue biopsy. Thus, a cumbersome process is required that performs a separate tissue biopsy on lesions which are suspicious of being cancer. In addition, although a biopsy may be performed, the accuracy of a diagnosis of cancer may be lowered depending on the doctor's professional skill.

### SUMMARY OF THE INVENTION

Thus, the present inventors conducted studies to solve the above problems occurring in the prior art. As a result, they developed a sensor for detecting cancerous tissue using needle electrodes, and found that normal tissue can be distinguished from cancerous tissue using the measurement of electrical signals obtained using the electrodes, that is, capacitance, thereby completing the present invention.

Accordingly, an object of the present invention is to provide a sensor for detecting cancerous tissue, including a board, one or more pairs of needle electrodes formed on the board to obtain electrical signals from tissue, and an output unit configured to output the electrical signals, obtained using the electrodes, to the outside, a method of manufacturing the same, and a method of monitoring the presence and status of cancerous tissue in real time.

In order to accomplish the above object, the present invention provides a sensor for detecting cancerous tissue, including a board; one or more pairs of needle electrodes formed on the board and configured to obtain electrical signals from tissue; and an output unit configured to output the electrical signals, obtained using the electrodes, and a method of manufacturing the same.

In addition, in order to accomplish the above object, the present invention provides a system for detecting cancerous tissue, including a sensor module configured to include a board, one or more pairs of needle electrodes formed on the board and configured to obtain electrical signals from tissue, and an output unit configured to output the electrical signals, obtained using the electrodes, to the outside; and, a processing module electrically connected to the output unit of the sensor module and configured to process the electrical signals output via the output unit.

In addition, in order to accomplish the above object, the present invention provides a method of monitoring the presence and status of cancerous tissue in real time, including attaching needle electrodes of the tissue sensor to a tissue site to be measured; and measuring capacitance between the needle electrodes in real time.

In addition, in order to accomplish the above object, the present invention provides a method of manufacturing a sensor for detecting cancerous tissue, including forming one or more pairs of needle electrodes on a board; and forming an output unit electrically connected to the electrodes and configured to output electrical signals.

In addition, in order to accomplish the above object, the present invention provides a method for manufacturing a sensor for detecting cancerous tissue in a form of a chip, including patterning a board by processing a portion of one side of the board with a non-conductive material; forming one or more pairs of needle electrodes on a remaining side of the board in a pattern identical to the above pattern; and forming an output unit by depositing a conductive material on the patterned board.

### BRIEF DESCRIPTION OF THE DRAWINGS

The above and other objects, features and advantages of the present invention will be more clearly understood from the following detailed description taken in conjunction with the accompanying drawings, in which:

FIG. 1 is an image of a sensor for detecting cancerous tissue according to the present invention;

FIG. 2 is a schematic view showing manufacture of an array sensor for detecting cancerous tissue;

FIG. 3 is a schematic view showing the results of taking a measurement using the sensor for detecting cancerous tissue in the form of a chip according to the present invention;

FIG. 4 is a schematic view showing the measurement methods for detecting cancerous tissue in the form of a chip according to the present invention;

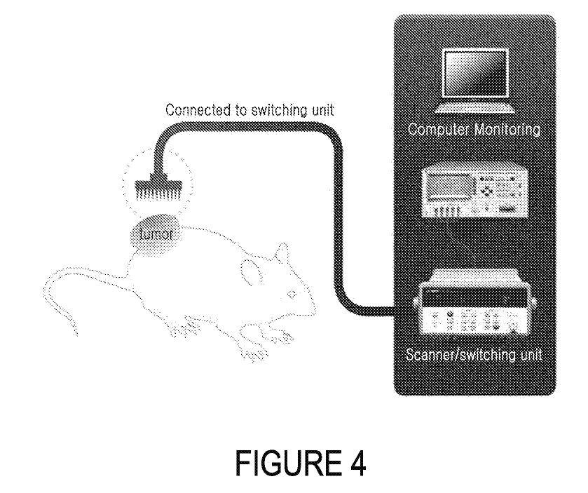

FIG. 5 is experimental results of capacitance which are measured as a function of frequency for normal and cancerous mouse tissue using the sensor according to the present invention;

FIG. 6 is experimental results illustrating the comparison of capacitance imaging of a mouse tissue using sensor for detecting cancerous tissue in the form of a chip according to the present invention with other cancer detecting methods—PET imaging and a histopathological tissue examination;

FIG. 7 is experimental results illustrating the capacitance imaging to detect the effect of an anticancer drug over time using the capacitance of mouse cancerous tissue and the sensor for detecting cancerous tissue in the form of a chip according to the present invention;

FIG. 8 is experimental results illustrating the capacitance image to detect growing of mouse cancerous tissue using the sensor for detecting cancerous tissue in the form of a chip according to the present invention; and

FIG. 9 is a schematic view illustrating an example in which the sensor for detecting cancerous tissue in the form of a chip according to the present invention is attached to an endoscope.

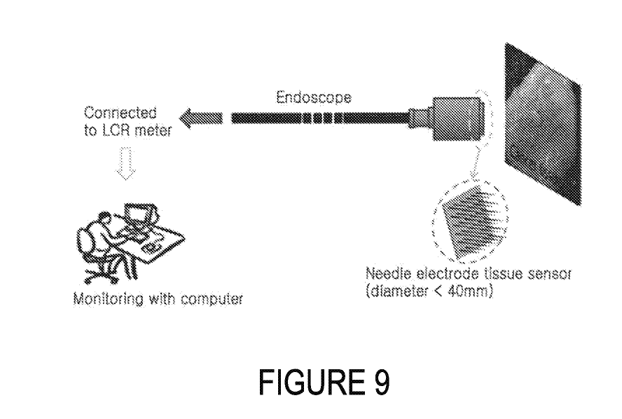

### DESCRIPTION OF THE PREFERRED EMBODIMENTS

Cancerous tissue has an electrical conductance which is 4 to 5 times higher than that of normal tissue, due to its high metabolic activity (Haemmerich D. et al., Physiol. Meas 24:251-60, 2003). In addition, the dielectric constant of cancerous tissue is also higher than that of normal tissue, and is not uniform (ANDRZEJ J. et al., IEEE TRANSACTIONS ON BIOMEDICAL ENGINEERING, 35: NO. 4, 1988). That is, cancer cell tissue may be considered a heterogeneous conductor, or an impurity in a conductor in terms of electricity. Based on this, normal tissue can be distinguished from cancerous tissue by measuring the difference in capacitance between those tissues.

The present invention relates to a sensor for detecting cancerous tissue, including a board; one or more pairs of needle electrodes formed on the board and configured to obtain electrical signals from tissue; and an output unit configured to output the electrical signals, obtained using the electrodes, to the outside.

The electrical signal may be capacitance.

The board is preferably one or more selected from the group consisting of a printed circuit board (PCB), a silicon board, and a polyimide board, but is not limited thereto.

The needles of the electrodes are preferably formed of one or more materials selected from the group consisting of silicon, gold, platinum, conductive polymer, and stainless steel, but are not limited thereto. The conductive polymer may include polypyrrole, polythiophene, and the like, but is not limited thereto.

The dimensions of the needles of the electrodes may be a diameter ranging from 0.5 μm to 1.5 mm (preferably from 0.5 μm to 1 mm) and a length ranging from 10 μm to 30 mm (preferably from 12 μm to 10 mm). When a dimension exceeds the ranges, there arises a problem, such as damage to cancerous tissue to be imaged, damage to adjacent normal tissue, or the possibility of departing from a range to be imaged.

Furthermore, in order to clearly identify the boundary between cancer and normal tissue, the intervals between electrodes may range from 1 μm to 3 mm (preferably from 1.5 μm to 1 mm).

In order to prevent the contamination of tissue when electrical signals are obtained, the needles may be packed up and disposed of, or may be used after being sterilized with ultraviolet light or in an autoclave.

The electrodes are paired in the form of a chip.

The sensor for detecting cancerous tissue, manufactured in the form of a chip as described above, has electrodes at close intervals, so that the size and shape of cancerous tissue can be imaged in more detail and the efficiency of the measurement of electrical changes can be maximized.

The present invention also relates to a system for detecting cancerous tissue, including a sensor module configured to include a board, one or more pairs of needle electrodes formed on the board and configured to obtain electrical signals from tissue, and an output unit configured to output the electrical signals, obtained using the electrodes, to the outside; and a processing module electrically connected to the output unit of the sensor module and configured to process the electrical signals output via the output unit.

The needle electrodes are electrically connected to the output unit of the sensor module for outputting the electrical signals to the outside, and the electrical signals output via the output unit are imaged using the processing module.

The processing module may process capacitance.

The present invention also includes a method of monitoring the presence and status of cancerous tissue in real time, including attaching needle electrodes of the tissue sensor to a tissue site to be measured; and measuring capacitance between the needle electrodes in real time.

In addition, the present invention relates to a method of manufacturing a sensor for detecting cancerous tissue, including forming one or more pairs of needle electrodes on a board; and forming an output unit electrically connected to the electrodes and configured to output electrical signals.

The present invention also relates to a method for manufacturing a sensor for detecting cancerous tissue in a form of a chip, including patterning a board by processing a portion of one side of the board with a non-conductive material; forming one or more pairs of needle electrodes on a remaining side of the board in a pattern identical to the above pattern; and forming an output unit by depositing a conductive material on the patterned board.

First, a portion of one side of a board is etched in a lattice structure, and then a non-conductive material is deposited thereon, thereby patterning the board.

In particular, the pairs of needle electrodes are patterned at intervals (preferably in a range of 1.5 μm to 1 mm) each of which is equal to or wider than a minimum interval which allows electrical signals to be obtained.

Any material may be used as the non-conductive material as long as electric current does not pass through the material, but specifically, it may be one or more selected from the group consisting of glass, polymethyl methacrylate (PMMA), non-conductive polymer, and silicon oxide.

One or more pairs of needle electrodes are formed on the other side of the board in a pattern identical to the above-described pattern.

In order for the electrodes to be connected to the processing module for processing the electrical signals output via the output unit, a conductive material is deposited on the patterned board, and then an output unit for outputting the electrical signals to the outside is formed.

The conductive material may be one or more selected from the group consisting of gold, platinum, silver, and conductive polymer, but is not limited thereto.

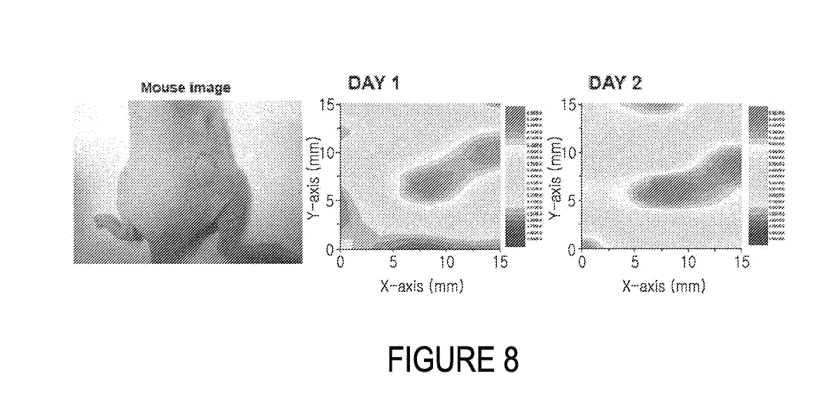

Since electrical signals (capacitance obtained using the sensor for detecting cancerous tissue may show cancerous tissue or the status (size and position) thereof in the form of an image, the boundary of a tumor may be determined more accurately. As a result, the removal of unnecessary normal tissue may be reduced during surgery using an endoscope. As shown in FIG. 8, the sensor may be used in the form of being attached to the head part of an endoscope.

That is, the sensor for detecting cancerous tissue according to the present invention may be used to diagnose as well as to treat and perform surgery on cancer.

Hereinafter, the present invention will be described in more detail with reference to embodiments and experimental examples. However, the following examples are provided for illustrative purposes only, and the scope of the present invention should not be limited thereto in any manner.

### Embodiment 1

### Manufacture of Sensor for Detecting Cancerous Tissue

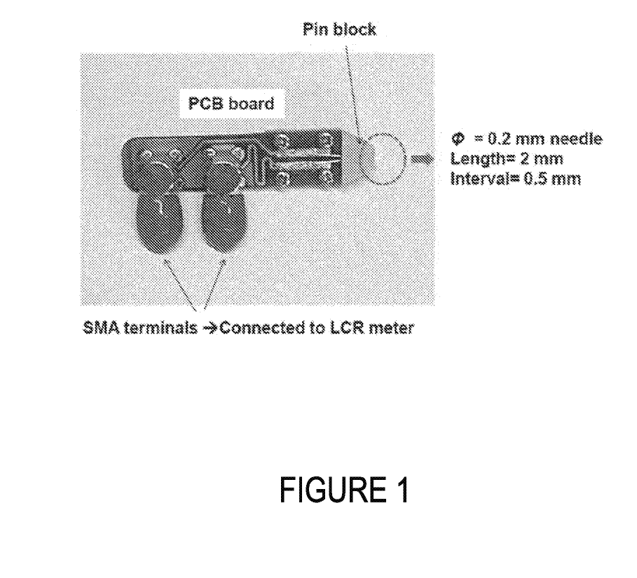

In order to take measurements on the cancerous tissue of a mouse, needles made of a stainless steel material were used. The dimensions of the needles were a diameter of 0.2 mm and a length of 2 mm. In order to stably position the needles in the cancerous tissue of the mouse while the capacitance was being measured, a pin block formed of a plastic material was manufactured and fixed the needles at an interval of 0.5 mm. Using this process, the needles could be maintained at the constant intervals and thus measurements were able to be taken over a long period of time. The fixed needles were electrically connected to a PCB board using silver glue. SMA terminals were connected to the PCB board, and the SMA terminals were connected to connectors that could be connected to the input terminals of an LCR (Inductance/Capacitance/Resistance) meter (see FIG. 1).

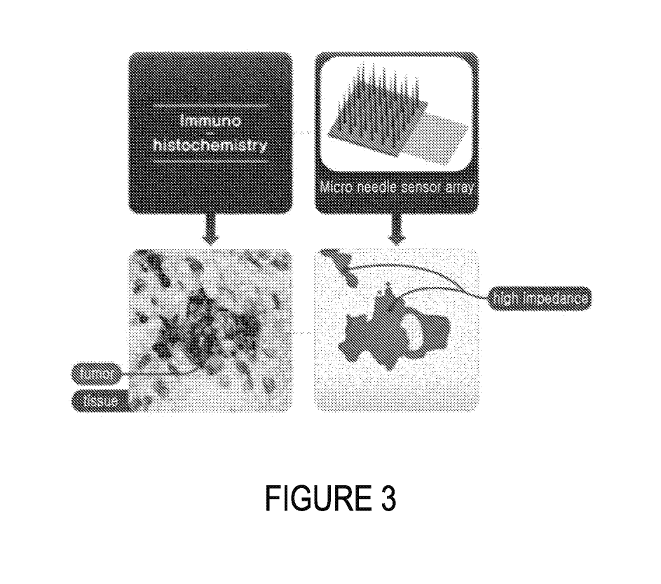

The measurement was performed by connecting a manufactured tissue sensor to the LCR meter as shown in FIG. 3 and the capacitance of the cancerous tissue of a mouse using a computer.

### Embodiment 2

### Manufacture of Sensor for Detecting Cancerous Tissue in the Form of Chip

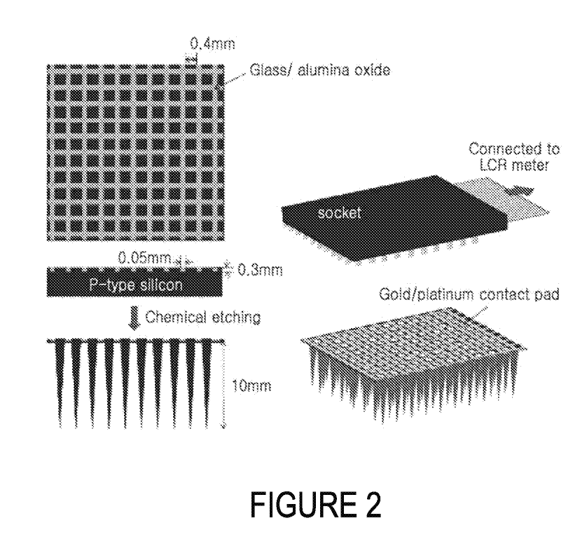

The rear side of a p-type silicon board with a width, length, and thickness of 42 mm, 42 mm, and 15 mm, respectively, was etched to form grooves with a width of 50 μm and a depth of 300 μm at intervals of 400 μm by using a diamond cutter, filled with glass in the form of powder, and then annealed at a temperature equal to or higher than 1000° C. in a furnace, thereby separating the silicon board at intervals of 400 μm. In addition, 10 mm needle-shaped electrodes are formed at intervals of 400 μm on the front side of the silicon board. Since each of the needle electrodes had to be connected to an LCR meter to take measurements, a gold thin film was deposited on the rear side of the separated silicon board and then the needle electrode was joined to a socket which was connected to the input terminals of the LCR meter. Using this process, 50 pairs of array sensors which each may operate as one capacitor can be manufactured (see FIGS. 2 and 3).

### Experimental Example 1

### Measurement of Capacitance Using Sensor for Detecting Cancerous Tissue

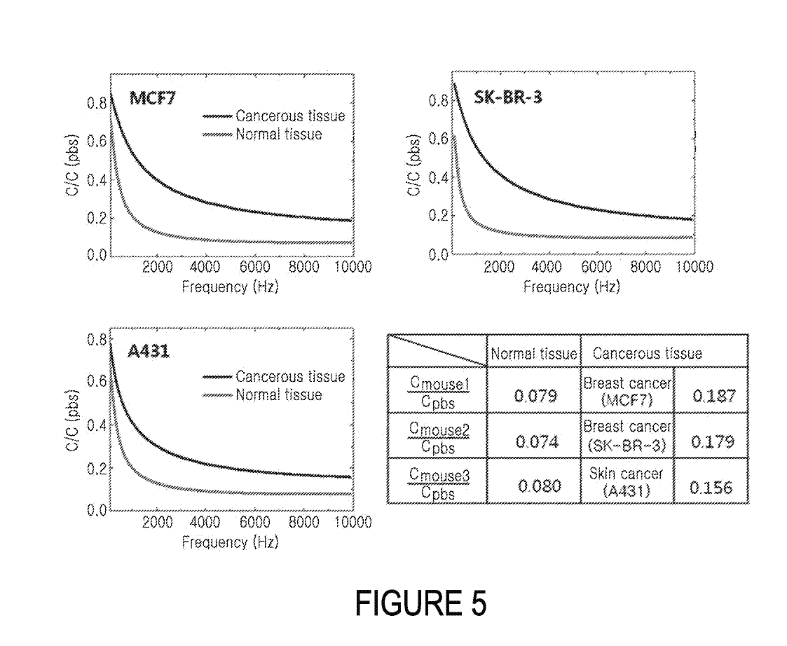

The tissue sensor of Embodiment 1 was attached to the cancerous tissue sites of a mouse in which MCF-7 and SK-BR-3 (breast cancer cell lines) and A431 (skin cancer cell line) had been grown, respectively, and the normal tissue site thereof, and then capacitance was measured by varying the frequency in a range from 100 Hz to 10 kHz. Here, a control experiment was performed by using a PBS buffer before the capacitance of each of the tissues was measured, and then capacitance values measured for the cancer and normal tissues were normalized using the results of the control experiment. In all of the three cases, the measurement showed that the capacitance of the cancerous tissue was higher than that of the normal tissue. Moreover, the measured value (0.156) of the skin cancer cell line A431 was significantly lower than those (0.187 and 0.179) of the breast cancer cell lines MFC-7 and SK-BR-3. In the same experiment, the measured values of normal tissues were 0.0079, 0.074 and 0.080, respectively, and the differences therebetween were lower than those with respect to cancerous tissues. When the tissue sensor of the present invention is used in this way, cancerous tissue can be clearly distinguished from normal tissue and the type of cancerous tissue can be also identified (see FIG. 5).

### Experimental Example 2

### Measurement of Capacitance with Sensor for Detecting Cancerous Tissue

FIG. 5 shows the results of measuring the capacitance of cancerous tissue, grown in a mouse, by using the sensor for detecting cancerous tissue, which is manufactured in the form of a chip in Embodiment 2.

Parts surrounded by red dotted lines are regions of cancerous tissue, while parts outside the dotted lines are regions of normal tissue. The measured region had a dimension of 15 mm×15 mm, and the arrangement of the sensor for detecting cancerous tissue, which was in the form of a chip and was used to take the measurement, was a 10×10 arrangement. As expected, cancerous tissue exhibited higher measured capacitance than normal tissue. The results of capacitance imaging showed the measured capacitance according to position.

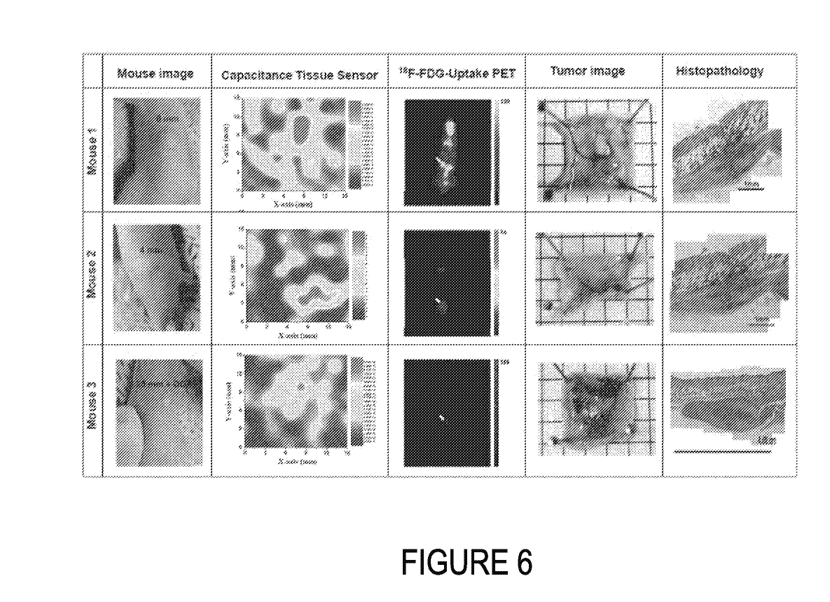

Cancerous tissue sites (red parts) showed higher values than normal tissue sites (blue parts), and the values of capacitance became smaller as the sensor was getting closer to the boundary between cancer and normal tissue. As a result of measuring the same site by using PET equipment which is typically used in medical cancer diagnosis, it was found that a cancerous tissue region(cancer size: less than 5 mm) indicated by a white arrow could not be clearly identified. From histological images based on the results of the imaging of removed mouse cancerous tissue and the results of a histopathological examination, it was found that cancerous tissue sites indicated by red regions were all cancerous tissues and their sizes were also less than 5 mm. Accordingly, the results of FIG. 6 show that the capacitance of cancerous tissue is higher than that of normal tissue and that the small size of cancerous tissue which is difficult to identify using PET can be imaged.

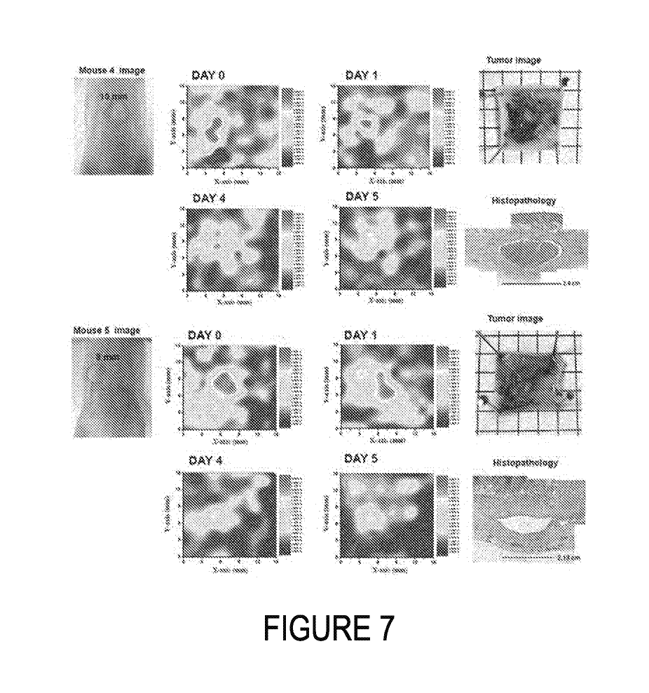

FIG. 7 shows the results which were obtained by growing cancerous tissue in a mouse as in the above experiment, injecting doxorubicin (100 ug/ml; 200 ul), used as an anticancer drug, into the cancerous tissue in the mouse, and imaging capacitance using the tissue sensor in the period from day 0 (injection day) to day 5. As a result, it was found that cancerous tissue regions indicated by the red color reduced more and more over time in mouse 4 and mouse 5 and the red color region disappeared on day 5. From the histological images based on the results of histopathology, it was found that cancer cells in cancerous tissue were destructed by doxorubicin in mouse 4 and mouse 5.

In contrast to FIG. 7, FIG. 8 shows the results of measurements of the situation where cancerous tissue formed in a mouse was growing. In FIG. 8, a red region which was a cancerous tissue site had been expanded.

The present invention relates to a sensor for detecting cancerous tissue using needle electrodes, which can obtain electrical signals, thereby distinguishing between cancer and normal tissues and imaging the status (size and position) of cancerous tissue. In addition, when the sensor for detecting cancerous tissue according to the present invention is attached to inspection equipment such as an endoscope, whether cancer has occurred can be determined within a short time by simply measuring changes in electrical signals without requiring the cumbersome work of performing a separate biopsy on a lesion. Furthermore, the difference in numerical data between cancer and normal tissue obtained from the results of the measurement of changes in electrical signals can be objectively and easily read irrespective of a doctor's professional skill, and thus the accuracy of the diagnosis of cancer can be improved.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A sensor for detecting cancerous tissue, comprising:
a board;
one or more pairs of needle electrodes formed on the board and configured to obtain electrical signals from tissue; and
an output unit configured to output the electrical signals, obtained from the electrodes, to an outside.

2. The sensor as set forth in claim 1, wherein the electrical signal is capacitance.

3. The sensor as set forth in claim 1, wherein the board is one or more selected from the group consisting of a printed circuit board (PCB), a silicon board, and a polyimide board.

4. The sensor as set forth in claim 1, wherein needles of the electrodes are formed of one or more materials selected from the group consisting of silicon, gold, platinum, conductive polymer, and stainless steel.

5. The sensor as set forth in claim 1, wherein dimensions of needles of the electrodes are a diameter ranging from 0.5 μm to 1.5 mm and a length ranging from 10 μm to 30 mm.

6. The sensor as set forth in claim 1, wherein intervals between the electrodes are in a range from 1 μm to 3 mm.

7. The sensor as set forth in claim 1, wherein the electrodes are paired in a form of a chip.

8. A system for detecting cancerous tissue, comprising:
a sensor module configured to comprise:
a board;
one or more pairs of needle electrodes formed on the board and configured to obtain electrical signals from tissue; and
an output unit configured to output the electrical signals, obtained using the electrodes, to an outside; and
a processing module electrically connected to the output unit of the sensor module and configured to process the electrical signals output via the output unit.

9. The system as set forth in claim 8, wherein the processing module processes capacitance.

10. A real-time method for monitoring presence and status of cancerous tissue, comprising:
attaching needle electrodes of the sensor of any one of claims 1 to 7 to a tissue site; and
measuring capacitance between the needle electrodes in real time.

11. A method for manufacturing a sensor for detecting cancerous tissue, comprising:
forming one or more pairs of needle electrodes on a board; and
forming an output unit electrically connected to the electrodes and configured to output electrical signals.

12. A method for manufacturing a sensor for detecting cancerous tissue in a form of a chip, comprising:
patterning a board by processing a portion of one side of the board with a non-conductive material;
forming one or more pairs of needle electrodes on a remaining side of the board in a pattern identical to the above pattern; and
forming an output unit by depositing a conductive material on the patterned board.

13. The method as set forth in claim 12, wherein the pattern has intervals in a range from 1 μm to 3 mm.

14. The method as set forth in claim 12, wherein the non-conductive material is one or more selected from the group consisting of glass, polymethyl methacrylate (PMMA), non-conductive polymer, and silicon oxide.

15. The method as set forth in claim 12, wherein the conductive material is one or more selected from the group consisting of gold, platinum, silver, and conductive polymer.

16. An endoscope comprising the sensor of any one of claims 1 to 7.
